(19) United States
(12) Patent Application Publication
(10) Pub. No.: US 2026/0363817 A1
(43) Pub. Date: Nov. 19, 2026

(54) KNEE ARTHROSCOPY LEG SUPPORT ARRANGEMENT

(71) Applicant: Tubantia Zeeland B.V., Bergen op Zoom (NL)

(72) Inventor: Jacob Siepko LUTTJEBOER, Bergen op Zoom (NL)

(21) Appl. No.: 19/487,720

(22) PCT Filed: May 31, 2024
(86) PCT No.: PCT/NL2024/050278

§ 371 Date: Nov. 25, 2025

(30) Foreign Application Priority Data
Jun. 2, 2023 (NL) 2034981

(51) Int. Cl. A61B 90/60

(52) U.S. Cl. CPC A61B 90/60

(57) ABSTRACT

A wearable knee arthroscopy leg support arrangement includes: a belt arranged to be worn by a user; a base part mounted to the belt, wherein the base part is arranged to envelop the belt in a direction perpendicular to a width of the belt; a protrusion, fixed to the base part and protruding radially outward from the belt during use, wherein the protrusion is arranged to support a leg, preferably a lower half of a leg, more preferably a part of a leg near an ankle; and wherein the protrusion includes a cushioning section.

### INCORPORATION BY REFERENCE TO ANY PRIORITY APPLICATIONS

Any and all applications for which a foreign or domestic priority claim is identified in the Application Data Sheet as filed with the present application are hereby incorporated by reference.

### FIELD

The invention relates to a wearable knee arthroscopy leg support arrangement.

### BACKGROUND

Knee arthroscopy is a type of surgical procedure used to diagnose and treat problems in the knee joint. This may involve inserting a small camera, the arthroscope, into the knee joint through small incisions. The camera provides a view of the inside of the joint on a monitor, allowing the medical personnel to see and diagnose problems related to the knee, and the knee joint in particular, such as torn ligaments, meniscus and cartilage damage.

Knee arthroscopy is usually performed on an outpatient basis, i.e. the patent can go home on the same day of the procedure. The procedure generally involves the steps making incisions in the patients knee, around the outer knee joint. Afterwards, the arthroscope can be inserted into the incisions. The camera of the arthroscope sends images, for example a video stream, of the inside of the knee joint to the monitor. In order to facilitate insertion of the arthroscope in to the knee joint, the surgeon may need to adjust position the knee joint. This can be done by moving the lower leg, i.e. the part below the knee towards to ankle, relative to the upper leg, i.e. the part above the knee towards the torso. This is conventionally done as follows. The patient lies at least with their upper body, e.g. from the hip upwards, on a horizontal surface such as a bed. The leg that comprises the knee that is to be examined, is supported near its lower section, e.g. near the ankle, on the hip or pelvis of a surgeon. The surgeon is than able to, during the procedure, to manipulate the position of the knee joint via movement of their hip.

A disadvantage of manipulating the knee joint via the hip of the surgeon is that this is uncomfortable for the surgeon, and may result in work related injuries such as lower back pain. Usually the pelvis is lifted on one side to prevent the lower leg/ankle from falling down. This is often done by the surgeon standing with one foot on a small, low bench. The surgeon may have to stand relative long times in an unnatural position, e.g. slanted to a side of the hip. Where in the past surgeons often removed parts of the meniscus or cartilage or cleaned the cartilage, meniscus and cartilage repairs are more commonly performed in the recent years. Repairing is a more challenging and time consuming procedure than cleaning or removing, putting an even greater strain on the surgeon.

### SUMMARY

The invention aims to counteract the above disadvantages, preferably while retaining the advantages. More specifically, the invention aims to provide for an arrangement that allows the surgeon to stand in a more natural position, e.g. a more comfortable position in which a longitudinal direction of the hip or pelvis of a surgeon is in a substantially horizontal direction, while still being able to manipulate the position of the knee joint.

Therefore, the invention provides for a wearable knee arthroscopy leg support arrangement, in particular the wearable knee arthroscopy leg support arrangement according to claim 1. The arrangement comprises a belt arranged to be worn by a user. The arrangement further comprises a base part mounted to the belt, wherein the base part is arranged to envelop the belt in a direction perpendicular to a width of the belt. Additionally, the arrangement comprises a protrusion fixed to the base part and protruding radially outward from the belt during use. The protrusion is arranged to support a leg, preferably a lower half of a leg, more preferably a part of a leg near an ankle. The protrusion comprises a cushioning section.

In the context of the invention the term ‘wearable’ should be understood as meaning that a user can equip the arrangement such that the arrangement is carried by the user. At the same time, the user should be able to move around with relative ease. Additionally, wearable should be understood to include that the support arrangement can be equipped and unequipped, e.g. like a piece of clothing.

The belt is arranged such that, when the arrangement is worn, the belt circumvents the waist of a user. Preferably, the belt can rest on the waist of a user. This may be facilitated by providing a belt buckle in the belt, such that the belt can be adjusted to the waist size of a user. As an additional advantage the belt buckle may allow for the belt to open, i.e. disconnecting a first end and a second end of the belt, such that a user may more conveniently equip the arrangement. In a preferred embodiment, the belt may be a relatively wide and thick belt in order to better facilitate the distribution of forces applied on the protrusion. A relatively thick and wide belt is known and used in other technical field, e.g. belts used for lifting weights during exercises such as dead lifting and squatting. From this field it is known that a relatively thick and wide belt allows for a better support of the core of the user, i.e. the torso.

The base part is arranged to envelope the belt. Enveloping the belt may facilitate efficient transfer of forces applied to the protrusion to the belt. For example, the forces applied to the protrusion during use may be in the direction that corresponds to the width of the belt. By arranging the base part such that it envelopes the belt in an orientation substantially in the same direction as the force applied on the protrusion, the forces are effectively transferred to the belt. A further advantage of enveloping the base part around the belt, instead of, for example, fixing the base part to the belt is that the base part can be moved by the user along the belt, e.g. towards the users front or back. This may allow the user to place the leg in an optimal position, while still standing in a natural, upright, position.

The base part is arranged such that, during use, it is provided on a side of the user, e.g. under a shoulder of the user, preferably between the shoulder and hip. Additionally or alternatively, the base part can be releasably mounted to the belt, e.g. using a bolts and nuts or a snap-fit connection. This may allow for a preferred placement of the base part, e.g. under the left shoulder or under the right shoulder. In an embodiment, a further base part and a further protrusion is provided such that a protrusion is provided under both shoulders such that the user can switch during the procedure on which side the leg rests.

The wearable knee arthroscopy leg support can further comprise a harness arranged to be worn by a use. The harness can comprise at least one shoulder strap arranged to be connected to the belt such that the belt is suspended from the at least one shoulder strap during use. Such a harness may facilitate the distribution of forces applies by the leg on the protrusion when more equally over the body of the person wearing the arrangement. Preferably, the at least one shoulder strap is provided on a shoulder of the user, e.g. a surgeon, opposite of the shoulder located on the side on which protrusion is provided. In a preferred embodiment, the harness can comprise a shoulder strap for each shoulder of the user. The two shoulder straps can each be attached to the belt, such that the belt can be suspended from the shoulder straps. The shoulder straps may be arranged vertically, i.e. such that when the belt is suspended from the shoulder straps, the straps are provided substantially vertical between the corresponding shoulder and the belt. Alternatively, the shoulder straps may be arranged diagonally, i.e. such that when the belt is suspended from the shoulder straps, the straps are provided substantially diagonally across the torso of the user. However, it will be clear to the person skilled in the art that any arrangement of shoulder straps can be provided, preferably such that the belt is suspended from the straps during and such that the forces applied on the protrusion are distributed by the harness.

The protrusion is fixed to the base part such that, during use, the protrusion points radially outward from the belt, i.e. away from the user. The protrusion has a cushioning section, such that a leg of a patient, preferably the ankle, can be placed on the protrusion without causing discomfort. This allows the surgeon to stand naturally, e.g. straight and not slanted to one side, while still being able to support the leg of the patient. In other words, the leg of a patient rests on the protrusion of the arrangement. The downward force caused by the leg is transferred to the belt via the base part, thereby distributing the force as a result of the weight of the patient's leg more equally over the body of the surgeon. Due to the location of the protrusion, the surgeon is still able to manipulate the knee-joint, e.g. by adjusting their upper body such that the protrusion moves the leg in a wanted position.

The base part can have a longitudinal extension extending below a width of the belt. Having a longitudinal extension of the base part below the width of the belt, preferably such that it abuts the hip of a user from the side, allows for the momentum and torque introduced by the weight of the leg in combination with the radially extending protrusion to be supported by the hip. This may provide for a better and more robust supporting of the leg by the arrangement, while minimizing the discomfort the momentum and torque may provide for the user. Additionally, the base part can be arranged to rest on a hip of a user when the wearable knee arthroscopy leg support arrangement is used. This may facilitate greater control for the user to manipulate the position of the knee-joint via the protrusion while allowing the vertical component of the force caused by the weight of the leg to be supported on the hip, while the hip is in a natural position while standing, e.g. upright.

The length of the protrusion in its longitudinal direction can be equal or less than the length of the base part in its longitudinal direction. Providing a protrusion that is equal or smaller than the length of the base part, in the direction it envelops the belt, it is ensured that the momentum and torque introduced by the weight of the leg in combination with the length of the protrusion in the radially outward direction is efficiently transferred via a relatively large surface to the user, thereby facilitating comfort provided by the arrangement during a knee arthroscopy.

The protrusion can have a length between 12 and 0.5 cm, preferably between 10 and 2 cm, more preferably between 8 and 4 cm, and even more preferably 7 cm, in its longitudinal direction. Providing a relatively small protrusion may allow the surgeon to wear the arrangement under the surgical clothing, e.g. a surgical gown, such that the arrangement can be covered by the surgical clothing such that medical hygiene can be maintained during the knee arthroscopy, as the patient or medical equipment will not be directly exposed to the leg support arrangement.

In a particular advantageous embodiment, the longitudinal extension is larger in its longitudinal direction than the length of the protrusion in its longitudinal direction. Or in other words, the longitudinal extension extends further below the belt, in a direction parallel to the width of the belt, than the protrusion extends radially outward from the belt. This configuration may more comfortably transfer the torque caused by the downward force exerted by a leg supported on the protrusion to the hip of the user during use, as the length of the arm of the protrusion is smaller than the length of the arm of the longitudinal extension.

The base part and the protrusion can be made out of rigid material, e.g. stainless steel or plastic. These materials are relatively light-weight while allowing the base part to be sufficiently stiff to more efficiently distribute the forces caused by the leg on the protrusion to the belt.

The protrusion may be arranged to be retractable or may be hingeable fixed to the base part, such that the most radially outward end of the protrusion can be moved to a position closer to a belt. This may be advantageous when a user is wearing the arrangement but does not need to support a leg, e.g. right before or after the operation. Then the protrusion most radially outward end can be moved closer to the belt, resulting in the protrusion protruding less compared to its original state and thus reducing the chance of accidental collusion or hindrance caused by the protrusion.

Further advantageous aspects of the invention are set out in the description and appended claims.

The technical features described in the paragraphs and sentences above can be isolated from the context, and the isolated technical features from the different paragraphs and sentences can be combined. Such combinations are herewith specifically disclosed in this description.

### BRIEF DESCRIPTION OF THE DRAWINGS

The invention will further be elucidated on the basis of exemplary embodiments which are represented in the drawings. The exemplary embodiments are given by way of non-limitative illustration of the invention.

In the drawings:

FIG. 1 shows a front view of a person wearing an example of a wearable knee arthroscopy leg support arrangement;

FIG. 2 shows an example of a base part and protrusion mounted to a belt of a wearable knee arthroscopy leg support arrangement;

FIG. 3 shows an example of two base parts and protrusions of a wearable knee arthroscopy leg support arrangement without cushioning sections; and

FIGS. 4A and 4B shows a front and rear view of a person wearing a second example of a wearable knee arthroscopy leg support arrangement respectively.

It is noted that the figures are only schematic representations that are given by way of non-limited examples. In the figures, the same or corresponding parts are designated with the same reference numerals.

### DETAILED DESCRIPTION

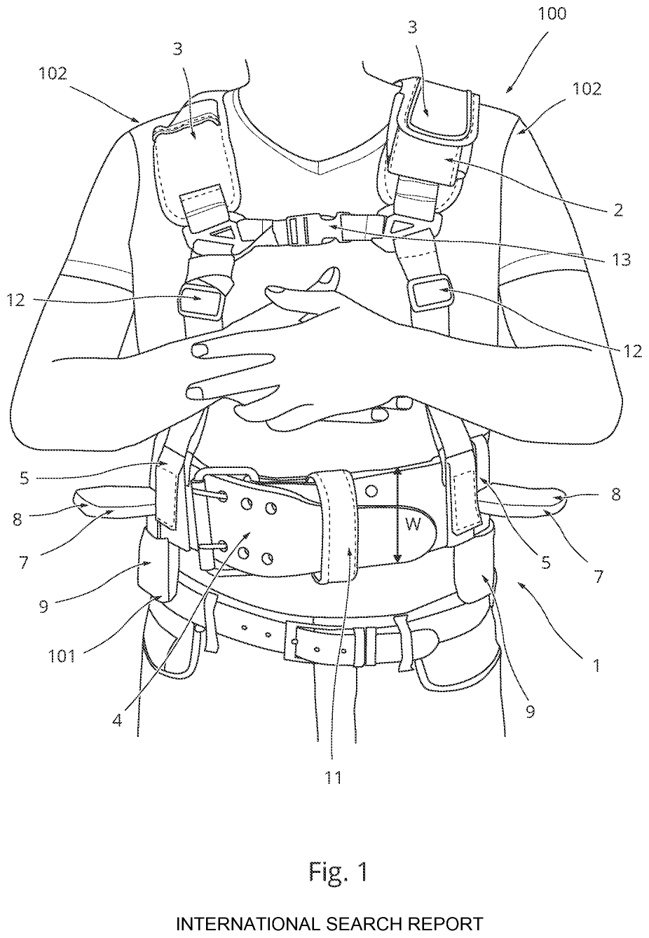

FIG. 1 depicts a front view of a user 100 wearing an example of a wearable knee arthroscopy leg support arrangement 1. In the example, the leg support arrangement 1 comprises a harness 2 arranged to be worn by the user 100. The harness 2 comprises two shoulder straps 3 and a belt 4 suspended from the two shoulder straps 3 during use. In the shown example, an additional strap 10 has been provided, connecting the two shoulder straps 3 to each other, ensuring a better fit and pretends the straps 3 from sliding down the shoulders 102 of the user 100. Preferably, such a strap 10 would provide a releasable connection between the two shoulder straps 2, for example via a snip-fit connection 13.

In order to provide a good fitting harness for the user, two different features are provided as examples. First, in order to adjust the circumferential length of the belt 4, a belt buckle 11 has been provided. Belt buckles are a known and commonly used feature to adjust a belt such that it can be worn comfortably, and fits, a user. Secondly strap adjusters 12 are have been provided on the front side of the harness 2. In the shown example, said strap adjusters 12 can be used to adjust the distance between the top of the shoulder straps 3, near the shoulders 102, and the belt 4. This may facilitate users to adjust the harness to fit comfortable, depending on the size of the user 100. For example, a tall user 100 may use the strap adjusters 12 to provide for relatively long shoulder strap 2, measured from the shoulders 102 to the belt 4, than a shorter user 100.

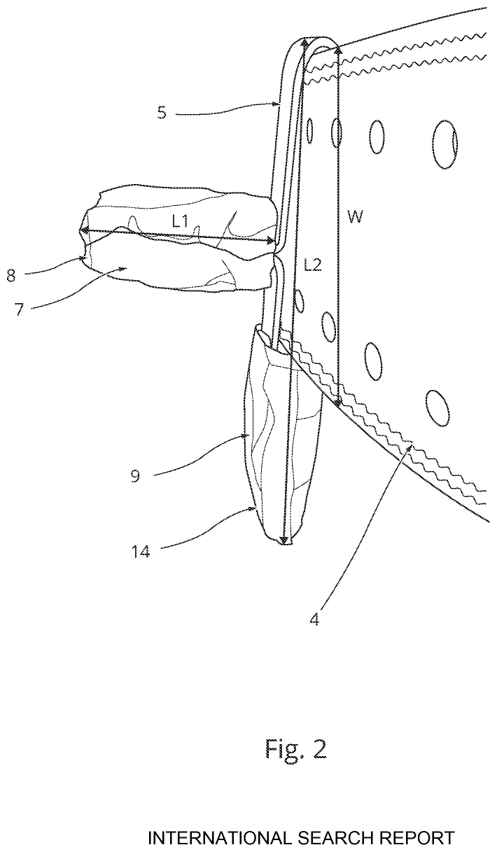

Turning to FIG. 2, a detailed example of the base part 5 and the protrusion 7 has been depicted when attached to the belt 4. The base part 5 is mounted to the belt 4, the base part 5 is arranged to envelop the belt 4 in a direction perpendicular to a width W of the belt 4. Additionally, the arrangement 1 comprises a protrusion 7 that is fixed to the base part 5 and protrudes radially outwardly from the belt 4. The protrusion 7 is arranged to support a leg (not shown), and comprises a cushioning section 8. In the shown example, the cushioning section 8 is made out of tape, providing already a cushioning effect on a leg of a patient (not depicted) resting on the protrusion 7. However, it will be clear that a better cushioning effect can be provided, for example using softer materials such as foam or rubber. Additionally or alternatively, it may be advantageous if the cushioning section 8 can be removed and replaced from the protrusion 7, for example by providing a tight fit between the cushioning section 8 and the protrusion using an elastic material. This can allow for the removal of the cushioning section 8. Additionally or alternatively, the cushioning section 8 may be attached to the protrusion 7 using Velcro, also allowing for the removal of the section 8 from the protrusion 7.

The base part 5 has a longitudinal extension 9, that extends below the width W of the belt 4. The base part 5 is further arranged to rest on a hip 101 of a user 100 during use. Preferably, and shown as an example, the longitudinal extension 9 comprises a second cushioning section 14 which may provide for comfort when the user 100 wears the supporting arrangement 1. The second cushioning section 14 may be constructed from the same materials as the cushioning section 8, or other materials.

The length L1 of the protrusion 7 is in its longitudinal direction less than the length L2 of the base part 5 in its longitudinal direction. Additionally, the protrusion 7 has a length of 7 cm in its longitudinal direction L1. Providing such a, relatively, small protrusion may allow the surgeon to wear the arrangement under the surgical clothing, e.g. a surgical gown, such that the arrangement can be covered by the surgical clothing such that medical hygiene can be maintained during the knee arthroscopy, as the patient or medical equipment will not be directly exposed to the leg support arrangement.

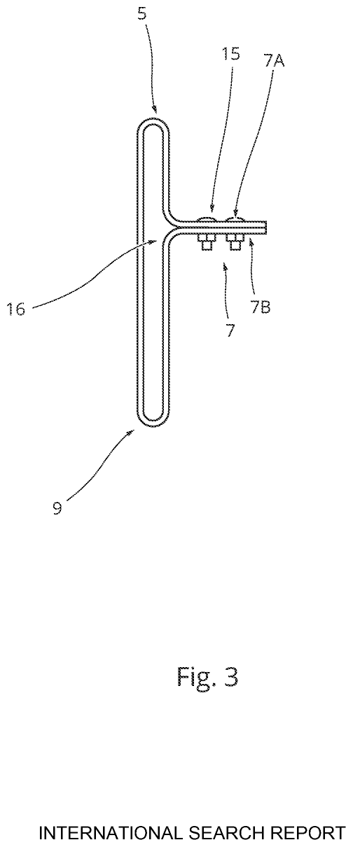

In a particular example, and as can be seen in FIG. 3, the longitudinal extension 9 is larger in its longitudinal direction than the length of the protrusion 7 in its longitudinal direction L1. Or in other words, the longitudinal extension 9 extends further below the belt 4, in a direction parallel to the width W of the belt 4, than the protrusion 7 extends radially outward from the belt 4. This configuration may more comfortably transfer the torque caused by the downward force exerted by a leg supported on the protrusion 7 to the hip of a user during use, as the length of the arm of the protrusion 7 is smaller than the length of the arm of the longitudinal extension 9.

Turning to FIG. 3, two base parts 5 and protrusions 7, without belt and cushioning sections 8, 14, have been depicted. From this example, a means has been provided to releasably attach the base part 5 to the belt. The base part 5 comprises two bolt/nut 15 combinations, which can be used to connect an upper protrusion section 7A to a lower protrusion section 7B, forming the protrusion 7 and a closed loop. Once the bolt/nut combinations 15 have been removed, the protrusion 7 can be split in to the upper section 7A and lower section 7B, allowing for the belt 4 to pass through, in to the opening 16 provided by the circumference of the base part 5.

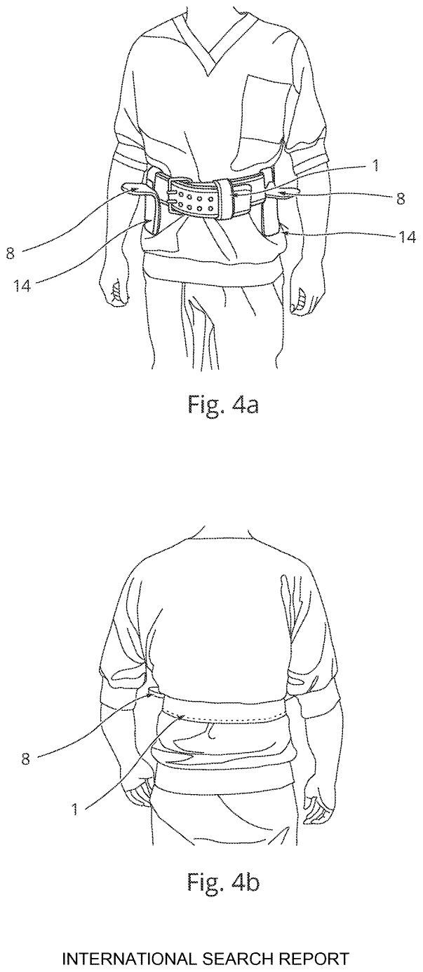

FIGS. 4A and 4B show a further example of a wearable knee arthroscopy leg support arrangement 1. In the shown example, the arrangement 1 is provided without the harness 2 of the first example depicted in FIG. 1. As an additional example, the cushioning sections 8, 14 are made from a neoprene and Velcro, which may provide for an even more comfortable experience for the surgeon and patient during use compared to the tape depicted in the first example.

Many variations will be apparent to the skilled person in the art. Such variations are understood to be comprised within the scope of the invention as defined in the appended claims. For example, it will be clear to the person skilled in the art that a harness is not needed, and that some users may prefer the use of a belt only. Additionally, different types of harnesses and belts may be used to distribute the forces applied by the leg of patient during use across the body of a user. Furthermore, it will be clear that any number of nut/bolt combinations or other fastening means can be used to connect the upper and lower protrusion section.

1. A wearable knee arthroscopy leg support arrangement comprising:
a belt arranged to be worn by a user;
a base part mounted to the belt, wherein said base part is arranged to envelop the belt in a direction perpendicular to a width of the belt; and
a protrusion, fixed to the base part and protruding radially outward from the belt during use,
wherein the protrusion is configured to support a leg, and wherein the protrusion comprises a cushioning section.

2. The wearable knee arthroscopy leg support arrangement according to claim 1, wherein the base part has a longitudinal extension extending below the width of the belt and wherein the base part is arranged to rest on a hip of a user when the wearable knee arthroscopy leg support arrangement is used.

3. The wearable knee arthroscopy leg support arrangement according to claim 1, wherein a length of the protrusion in its longitudinal direction is equal or less than length of the base part in its longitudinal direction.

4. The wearable knee arthroscopy leg support arrangement according to claim 1, wherein the base part and the protrusion are made of a rigid material.

5. The wearable knee arthroscopy leg support arrangement according to claim 1, wherein the protrusion has a length between 12 and 0.5 cm in its longitudinal direction.

6. The wearable knee arthroscopy leg support arrangement according to claim 1, further comprising a harness arranged to be worn by a user, wherein the harness comprises at least one shoulder strap arranged to be connected to the belt such that the belt is suspended from the at least one shoulder strap during use.

7. The wearable knee arthroscopy leg support arrangement according to claim 1, wherein the protrusion is configured to support a lower half of the leg.

8. The wearable knee arthroscopy leg support arrangement according to claim 1, wherein the protrusion is configured to support a part of the leg near an ankle.

9. The wearable knee arthroscopy leg support arrangement according to claim 4, wherein the base part and the protrusion are made of a stainless steel or a plastic.

10. The wearable knee arthroscopy leg support arrangement according to claim 1, wherein the protrusion has a length between 10 and 2 cm in its longitudinal direction.

11. The wearable knee arthroscopy leg support arrangement according to claim 1, wherein the protrusion has a length between 8 and 4 cm in its longitudinal direction.

12. The wearable knee arthroscopy leg support arrangement according to claim 1, wherein the protrusion has a length of 7 cm in its longitudinal direction.
